(19) United States
(12) Patent Application Publication
(10) Pub. No.: US 2009/0246188 A1
(43) Pub. Date: Oct. 1, 2009

(54) Method for Production of a Bioengineered Form of Tissue Plasminogen Activator

(76) Inventor: Villoo Morawala Patell, Karnataka (IN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/914,753

(22) PCT Filed: May 31, 2006
(86) PCT No.: PCT/IB06/01481

§ 371 Date: Apr. 21, 2008

(30) Foreign Application Priority Data
Jun. 2, 2005 (IN) 673/CHE/2005

(51) Int. Cl. A61K 38/48; C12N 15/09; A61P 35/00

(52) U.S. Cl. 424/94.64; 435/69.2

(57) ABSTRACT

The present invention relates to the recombinant method used for the production of soluble form of human tissue plasminogen activator variant. In this variant the threonine at position 103 of the endogenous tissue plasminogen activator is replaced by an asparagine leading to a new glycosylation site. At position 117 of the endogenous tissue plasminogen activator asparagine has been replaced by glutamine, leading to the removal of an N linked glycosylation site. At position 296-299 the amino acids lysine, histidine, arginine, and arginine have been replaced by four alanine amino acids. The invention further relates to the de novo synthesis of the nucleic acid sequence encoding tissue plasminogen activator, transformation of the constructed nucleic acid sequences into competent bacteria and sub-cloning of the same into mammalian expression vectors for the expression of the desired protein. DNA constructs comprising the control elements associated with the gene of interest have been disclosed. The recombinant human tissue plasminogen activator, according to the invention, and the salts and functional derivatives thereof, may comprise the active ingredient of pharmaceutical compositions for treatment of treatment of heart attack and stroke patients. These compositions are yet another aspect of the present invention.

### FIELD OF INVENTION

The present invention relates to the recombinant method used for the production of soluble form of human tissue plasminogen activator variant. In this variant the threonine at position 103 of the endogenous tissue plasminogen activator is replaced by an asparagine leading to a new glycosylation site. At position 117 of the endogenous tissue plasminogen activator asparagine has been replaced by glutamine, leading to the removal of an N linked glycosylation site. At position 296-299 the amino acids lysine, histidine, arginine, and arginine have been replaced by four alanine amino acids.

The invention further relates to the de novo synthesis of the nucleic acid sequence encoding tissue plasminogen activator, transformation of the constructed nucleic acid sequences into competent bacteria and sub-cloning of the same into mammalian expression vectors for the expression of the desired protein.

DNA constructs comprising the control elements associated with the gene of interest have been disclosed.

The recombinant human tissue plasminogen activator, according to the invention, and the salts and functional derivatives thereof, may comprise the active ingredient of pharmaceutical compositions for treatment of treatment of heart attack and stroke patients. These compositions are yet another aspect of the present invention.

### BACKGROUND OF INVENTION

Plasminogen activators are enzymes that activate the zymogen plasminogen to generate the serine proteinase plasmin, which degrades fibrin. Among the plasminogen activators studied are streptokinase, urokinase and human tissue plasminogen activator (t-PA). The mechanism of action of each of these plasminogen activators differs. Streptokinase forms a complex with plasminogen generating plasmin activity, urokinase cleaves plasminogen directly and t-PA forms a ternary complex with fibrin and plasminogen, leading to plasminogen activation in the locality of the clot.

Tissue type plasminogen activator (t-PA) a multidomain, glycosylated, serine protease is a fibrin specific activator of plasminogen and a very effective thrombolytic agent. t-PA is a recombinant protein whose primary application is in the treatment of heart attack and stroke patients. First characterized in 1979, as an important and potent biological pharmaceutical agent in the treatment of various vascular diseases due to its high fibrin specificity and potent ability to dissolve blood clots in vivo.

Natural t-PA has a plasma half-life of about six minutes or less. Due to its rapid clearance from the circulation, t-PA has to be infused to achieve thrombolysis. Front loaded dosing with increased concentrations of t-PA has shown more rapid and complete lysis compared to the standard infusion protocol and early potency is correlated with improved survival rate. Bolus administration could further improve the lytic rate by quickly exposing the target clot to a higher concentration of the enzyme, but single bolus administration of natural or wild type (wt) t-PA cannot be generally used, due its clearance rate.

Many investigators have produced longer half-life versions of t-PA that could be administered as a bolus, but almost all of the variants turned out to have significantly decreased fibrinolytic activities.

Thus it is an object of the present invention to provide recombinant method used for the production of a molecule with reduced clearance rate while retaining full fibrinolytic activity, systematic mutagenesis studies was applied to t-PA on its various domains. Such a drug would also have a high specificity with greater affinity for a recent thrombus and would produce less circulating plasmin. Consequently, the incidence of ICH and other non-cerebral bleeding events would be lower. The drug would have resistance to PAI-1 and also be cost effective.

### SUMMARY OF THE INVENTION

The present invention relates to the recombinant method used for the production of soluble form of human tissue plasminogen activator variant. In this variant the threonine at position 103 of the endogenous tissue plasminogen activator is replaced by an asparagine leading to a new glycosylation site. At position 117 of the endogenous tissue plasminogen activator asparagine has been replaced by glutamine, leading to the removal of an N linked glycosylation site. At position 296-299 the amino acids lysine, histidine, arginine, and arginine have been replaced by four alanine amino acids.

A particular aspect of the invention relates to de novo synthesis of the nucleic acid sequence encoding tissue plasminogen activator, transformation of the constructed nucleic acid sequences into competent bacteria and sub-cloning of the same into mammalian expression vectors for the expression of the desired protein.

Yet another aspect of the invention provides novel biologically functional vital and circular plasmid DNA vectors incorporating DNA sequences of the invention and host organisms stably transformed or transfected with said vectors.

Correspondingly provided by the invention are novel methods for the production of useful polypeptides comprising cultured growth of such transformed host cells particularly mammalian cells under conditions facilitative of large scale expression of the exogenous, vector-borne DNA-sequences and isolation of the desired polypeptides from the growth medium, cellular lysates or cellular membrane fractions.

### DETAILED DESCRIPTION OF FIGURES AND SEQUENCES

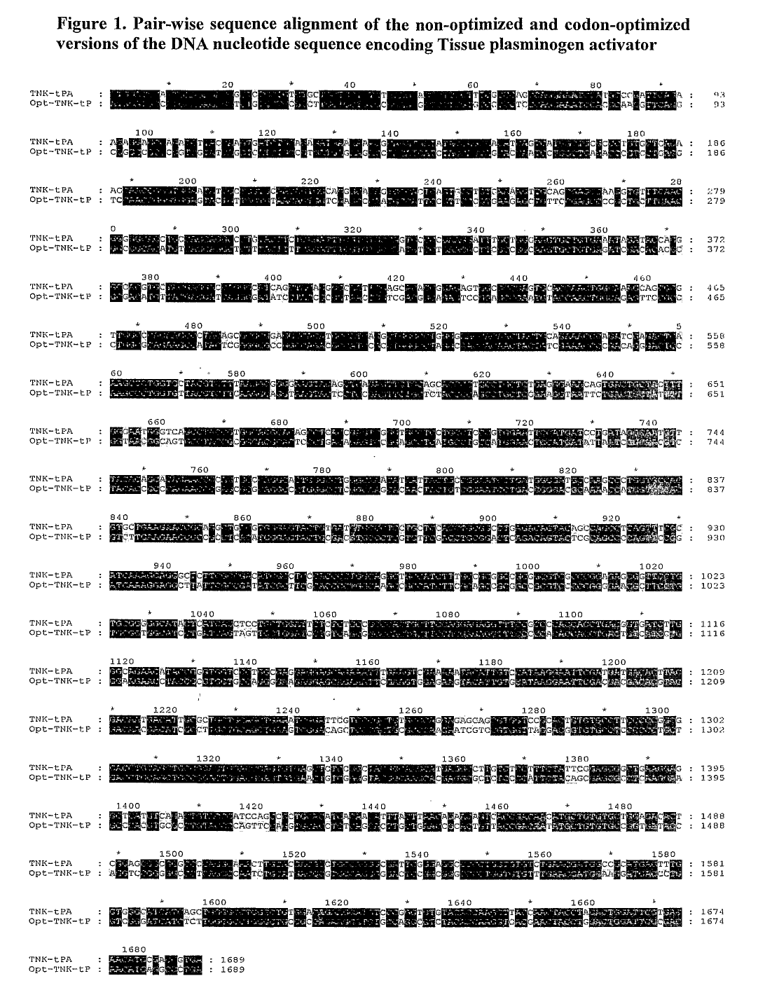

FIG. 1. Pair-wise sequence alignment of the non-optimized and codon-optimized versions of the DNA nucleotide sequence encoding Tissue plasminogen activator

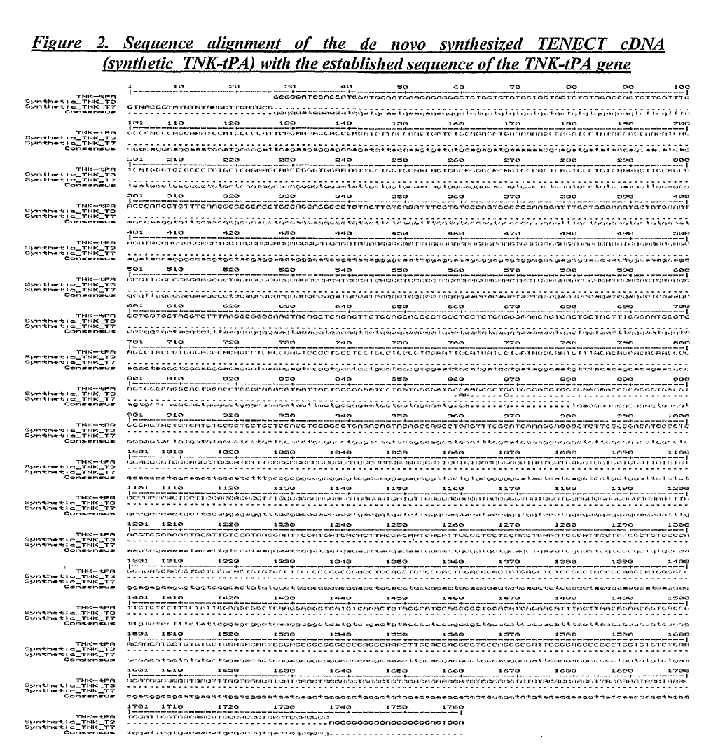

FIG. 2. Sequence alignment of the de novo synthesized TENECT cDNA (synthetic_TNK-tPA) with the established sequence of the TNK-tPA gene

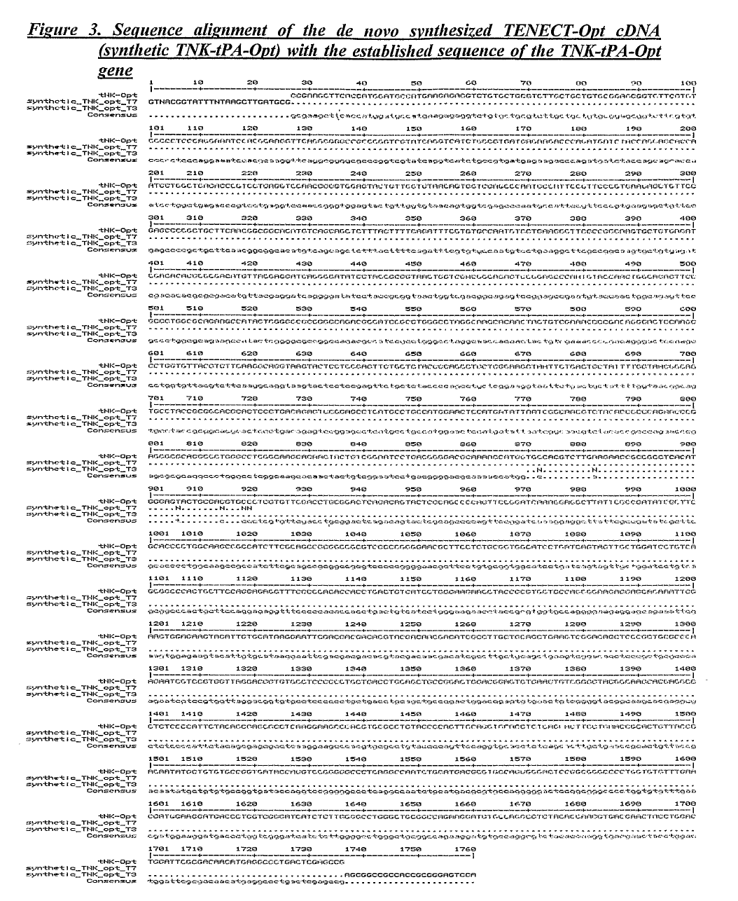

FIG. 3. Sequence alignment of the de novo synthesized TENECT-Opt cDNA (synthetic TNK-tPA-Opt) with the established sequence of the TNK-tPA-Opt gene

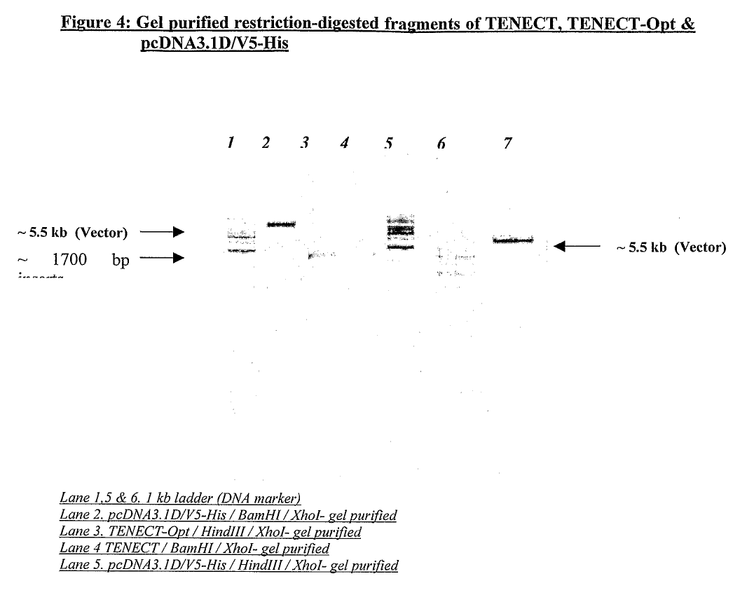

FIG. 4: Gel purified restriction-digested fragments of TENECT, TENECT-Opt & pcDNA3.1D/V5-His

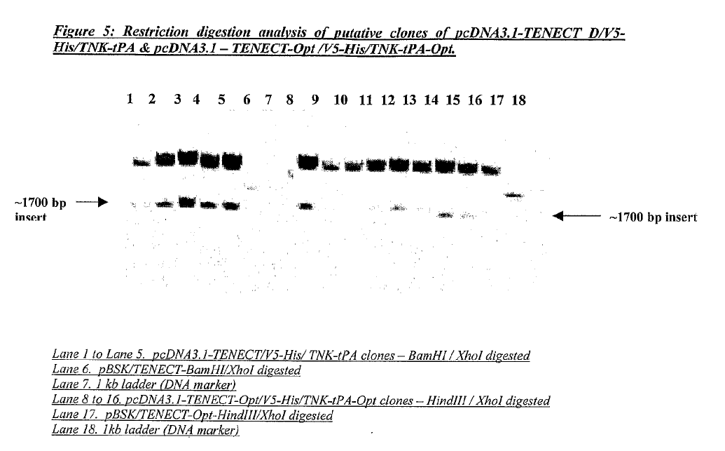

FIG. 5: Restriction digestion analysis of putative clones of pcDNA3.1-TENECT D/V5-His/TNK-tPA & pcDNA3.1—TENECT-Opt/V5-His/TNK-tPA-Opt.

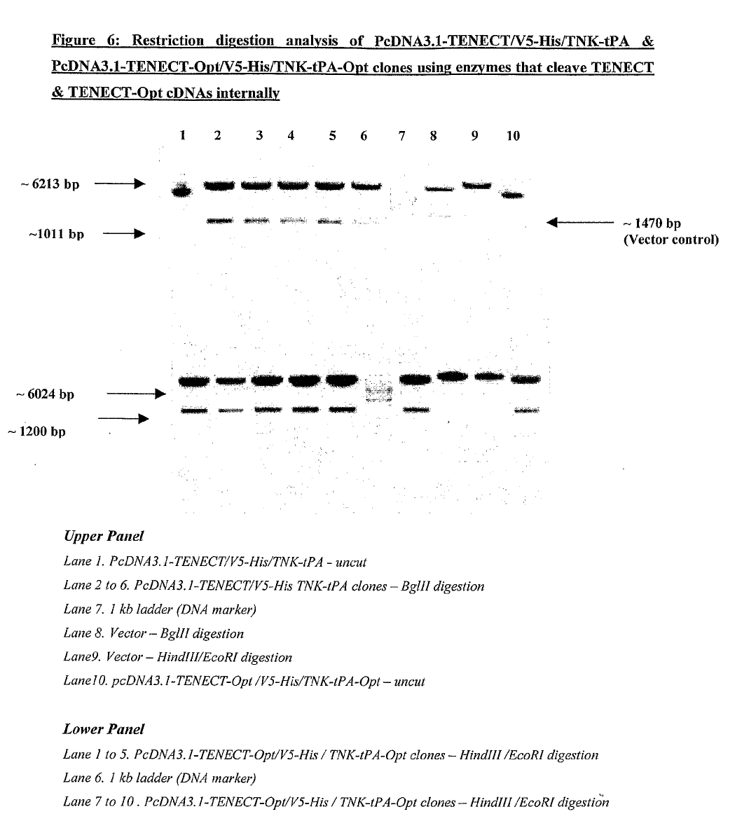

FIG. 6: Restriction digestion analysis of PcDNA3.1-TENECT/V5-His/TNK-tPA & PcDNA3.1-TENECT-Opt/V5-His/TNK(-tPA-Opt clones using enzymes that cleave TENECT & TENECT-Opt cDNAs internally

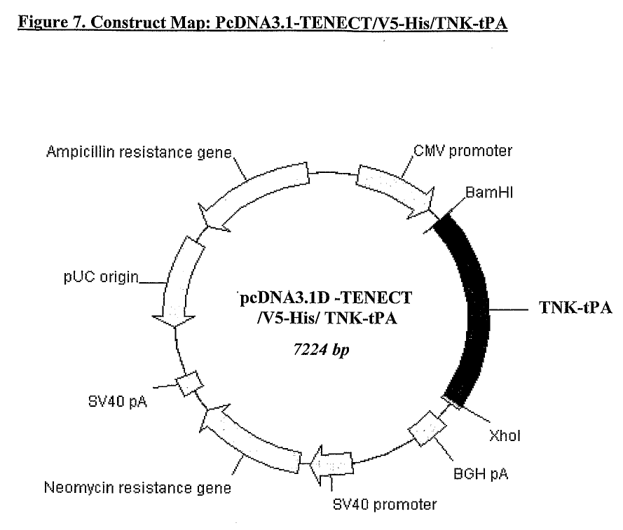

FIG. 7. Construct Map: PcDNA3.1-TENECT/V5-His/TNK-tPA

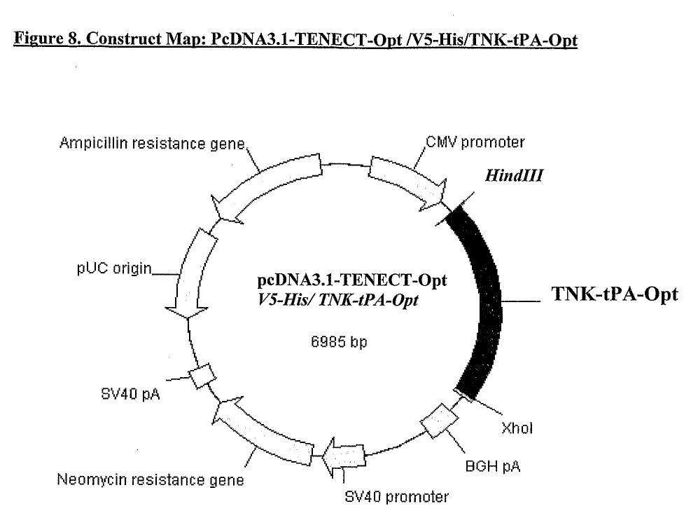

FIG. 8. Construct Map: PcDNA3.1-TENECT-Opt/V5-His/TNK-tPA-Opt

SEQ ID. No. 1. Nucleotide sequence encoding the recombinant tissue plasminogen activator

SEQ ID. No. 2. Codon-optimized version of the nucleotide sequence encoding the recombinant tissue plasminogen activator

### DETAILED DESCRIPTION OF THE INVENTION

Several methods have been described for the expression of recombinant proteins in higher eukaryotic systems. CHO-K1, HEK-293 (and variants) cell expression systems have now established themselves as the predominant systems of choice for mammalian protein expression. Refinements of vector construction, choice of selectable markers and advances in gene-targeting and high-throughput screening strategies have made the establishment of recombinant cell lines with high specific productivities relatively common and have reduced the time required for cell line development. Recent advancements in expression technologies using traditional viral-promoter-based expression vectors include the development and refinement of bi-cistronic expression strategies using either internal ribosome entry site (IRES) sequences or alternative splicing.

### Example 1

DNA sequences encoding tissue plasminogen activator were synthesized by de novo approach. This approach enables better codon optimization with respect to the particular mammalian cell line to be used. Further the synthetic DNA was made the subject of eucaryotic/prokaryotic expression providing isolatable quantities of polypeptides displaying biological properties of naturally occurring t-PA as well as both in vivo and invitro biological activities of t-PA.

Nucleotide sequence encoding the recombinant tissue plasminogen activator (TENECT 1) has been represented in the SEQ ID. No. 1. The codons in the coding DNA sequence of the tissue plasminogen activator that have been altered as part of the codon-optimization process to ensure optimal recombinant protein expression in mammalian cell lines such as CHO K1 and HEK 293 have been highlighted in uppercase. SEQ ID. No. 2 represents codon optimized nucleotide sequence encoding tissue plasminogen activator (TENECT 2)

Pair wise sequence alignment of the non-optimized and codon optimized nucleotide sequence encoding tissue plasminogen activator has been represented in FIG. 1.

### Example 2

### Verification of Authenticity of De Novo Synthesized cDNA Encoding Tissue Plasminogen Activator

The verification of the authenticity of the de novo synthesized cDNA molecules as supplied by the commercial service provider was done by automated DNA sequencing and the results obtained are depicted in FIGS. 2 & 3.

### Example 3

### Sub-Cloning of TENECT & TENECT-Opt cDNAs into the pcDNA3.1D/V5-His Mammalian Cell-Specific Expression Vector

Subsequent to the verification of the authenticity of the de novo synthesized cDNA molecules (TENECT & TENECT-Opt) by automated DNA sequencing as shown above. TENECT & TENECT-Opt were individually sub-cloned into the mammalian cell-specific expression vector pcDNA3.1D/V5-His to generate the transfection-ready constructs. The details of the procedures used are given below:

A. Reagents and enzymes:
1. QIAGEN gel extraction kit & PCR purification kit
2. pcDNA 3.1D/V5-His vector DNA (Invitrogen)

| Enzyme | Supplier | U/μl | 10x buffer |
|---|---|---|---|
| 1. BamHI | Bangalore Genei | 10 | Buffer E |
| 2. XhoI | Bangalore Genei | 10 | Buffer E |
| 3. HindIII | Bangalore Genei | 20 | Buffer E |
| 4. XhoI | Bangalore Genei | 10 | Buffer E |
| 5. T4 DNA ligase | Bangalore Genei | 40 | Ligase Buffer |

All reactions were carried out as recommended by the manufacturer. For each reaction the supplied 10× reaction buffer was diluted to a final concentration of 1×.

B. Restriction digestion of the vector and the insert:

Procedure

The following DNA samples and restriction enzymes were used:

| DNA samples | Restriction Enzyme |
|---|---|
| Rxn # 1 Vector (for TNK-tPA cloning) | BamHI/XhoI |
| Rxn # 2 Vector (for TNK-tPA-Opt cloning) | HindIII/XhoI |
| Rxn # 3 pBSK/TNK-tPA (#5) | BamHI/XhoI |
| Rxn # 4 pBSK/TNK-tPA-Opt (#18) | HindIII/XhoI |

Restriction enzyme digest reaction:

| Components | Final conc. | Rxn #1 | Rxn #2 | Rxn #3 | Rxn #4 |
|---|---|---|---|---|---|
| Water | — | 2 μl | 2 μl | 2 μl | 9 μl |
| 10x Buffer | 1x | 2 μl | 2 μl | 2 μl | 2 μl |
| DNA | — | 12 μl | 12 μl | 12 μl | 5 μl |
| BamHI | 0.5 U | 1 μl | — | 1 μl | — |
| XhoI | 0.5 U | 1 μl | — | 1 μl | — |
| HindIII | 1.0 U | — | 1 μl | — | 1 μl |
| XhoI | 0.5 U | — | 1 μl | — | 1 μl |
| 10x BSA | 1x | 2 l | 2 μl | 2 μl | 2 μl |
| Final volume | 20 μl | 20 μl | 20 μl | 20 μl | 20 μl |

The reaction was mixed, spun down and incubated for 2 hrs at 37° C. The restriction digestion was analysed by agarose gel electrophoresis. The expected digestion pattern was observed that featured a gene fragment fall out of 1700 bp (for Rxn # 3 & 4) and a vector backbone fragment of ˜5.5 kb for Vector (Rxn # 1 & 2) was seen. The ˜1700 bp DNA fragments representing TENECT & TENECT-Opt cDNAs were separately purified by the gel extraction method using the QIAGEN gel extraction kit. The ˜5.5 kb digested vector backbone of the pcDNA3.1D/V5-His mammalian expression vector was also purified using the same kit. Subsequent to the restriction digestion and gel-extraction of the requisite cDNA and vector DNA fragments, an aliquot (1-2 micro liter) of each purified DNA sample was analyzed using agarose gel electrophoresis to check for purity and integrity as shown in FIG. 4 below:

C. Ligation of pcDNA3.1D/V5-His backbone with TENECT & TENECT-Opt cDNAs:

The DNA concentration of the digested & purified vector and insert fragments was estimated (ref. FIG. 4 above) and ligation was set up in the following manner:

| | | | | | Rxn #4 |
|---|---|---|---|---|---|
| | | Rxn #1 | Rxn #2 | Rxn #3 | (T-Opt- |
| Components | Final conc. | (T-V) | (T-V + I) | (T-Opt-V) | V + I) |
| Water | — | 15 μl | 10 μl | 15 μl | 9 μl |
| 10x Rxn | 1 | 2 μl | 2 μl | 2 μl | 2 μl |
| Buffer | | | | | |
| Vector | 50 ng | 2 μl | 2 μl | 2 μl | 2 μl |
| Insert | 10 ng/8 ng | — | 5 μl | — | 6 μl |
| T4 DNA | 15 U | 1 μl | 1 μl | 1 μl | 1 μl |
| Ligase | | | | | |
| Final volume | 20 μl | 20 μl | 20 μl | 20 μl | 20 μl |

The reactions were gently mixed, spun down and incubated at R.T, 2-3 hrs. DH10 competent cells were transformed with the contents of ligation reaction mixtures.

D. Restriction digestion analysis of putative clones of pcDNA3.1-TENECT/V5-His/TNK-tPA & pcDNA3.1D-TENECT-Opt/V5-His/TNK-tPA-Opt.

Plasmid DNA was individually purified from the colonies obtained on L.B agar plates containing ampicillin and the presence of the desired cDNA insert was confirmed by restriction digestion analysis of the isolated plasmid DNA as shown in FIG. 5.

In accordance with the results obtained after the restriction digestion of several putative clones containing the pcDNA3.1-TENECT/V5-His/TNK-tPA & pcDNA3.1-TENECT-Opt/V5-His/TNK-tPA-Opt, some of the clones which showed the desired restriction pattern were selected for further restriction digestion analysis using restriction enzymes that cleave the TENECT & TENECT-Opt cDNAs internally to generate variable sized fragments as shown in FIG. 6.

Most of the PcDNA3.1-TENECT/V5-His/TNK-tPA & PcDNA3.1-TENECT-Opt/V5-His/TNK-tPA-Opt clones selected for the restriction mapping analysis yielded the expected fragment sizes based on the occurrence of known internal restriction sites and hence these clones will be further verified by DNA sequencing analysis.

The maps of the recombinant expression constructs made using the de novo synthesized TENECT and TENECT-Opt cDNAs are pictorially represented in the FIGS. 7 & 8.

### Example 4

### Maintenance and Propagation of the Human t-PA Construct

The maintenance and propagation of the cDNA construct encoding human t-PA will be done in standard bacterial cultures. Glycerol stocks of all the clones would be maintained and stored at −-70° C.

### Example 5

### Transient & Stable Recombinant Protein Expression in CHO-K1 Cells

Transient & stable expression of human t-PA was done using the Chinese hamster ovary cells (CHO), a mammalian cell line that has FDA approved for producing therapeutic proteins. Transient expression is useful to check the expression of a construct and to rapidly obtain small quantities of a recombinant protein.

The stable transfectants were screened for the expression of t-PA using tools like in vitro bioassay or ELISA and the best producer will be selected. Homogenous stable cell lines would be selected by clonal dilution and then amplified and frozen.

The protein expression would be further analyzed using analytical tools such as Western Blot, ELISA, and functional assays.

### Example 6

### Purification of Recombinant Tissue Plasminogen Activator

Subsequent to the establishment of a contaminant-free cell line, as per the guidelines of the regulatory agencies, that over-expresses the desired recombinant protein, the purification strategies will aim at process economics, speed to market, scalability, reproducibility, and maximum purity of the product with functional stability and structural integrity as the major objectives. To this effect, a combinatorial approach with both filtration (normal and tangential flow filtration) and chromatography would be explored. The process qualification requirements and acceptance criteria studies will be conducted on 3 batches.

Accordingly, the current invention envisages the following steps in the purification process and or of standard methods known per se:

a. Initial clarification and concentration of crude culture broth using normal and tangential flow filtration procedures

b. Ultra filtration/Dialysis filtration (based on tangential flow filtration)

c. Chromo step—I: Affinity chromatography using heparin, lysine, metal (Zinc) chelate Sepharose and mabs immobilized to Sepharose. More preferably, lysine Sepharose will be used in the downstream unit operations.

e. Chromo step—II: Anion exchange chromatography using DEAE cellulose

f. Virus removal and sterile filtration

g. Endotoxin removal

Note: Additionally, flow through based anion exchangers such as cellufine sulfate will be used for selective binding of process contaminants, endogenous/adventitious viruses and column extractables.

### Example 7

### Establishment of the Identity of the Target Protein Using Biochemical, Immunological and Physico-Chemical Methods

The percent recovery of the total protein at each stage will be quantitated using bicinchoninic acid procedure (BCA)/Bradford dye binding method. The target protein concentration will be routinely determined at each stage of purification using highly specific and reliable enzyme based immunoassays such as capture ELISA using polyclonal/monoclonal anti tPA antibodies standardized to native—sequence t-PA. Qualitative and target specific western analysis will be followed at each stage. Reversed phase chromatography, isoelectric focusing and two-dimensional gel electrophoresis will be employed to evaluate the purified product. Secondary structural analysis would be examined using far UV circular dichroism. Molecular mass and oligomeric status will be investigated using size exclusion and MALDI-TOF. The investigations will also focus on the stability of the protein in relation to pH and temperature.

1. A process for the preparation of an in vivo biologically active Tissue Plasminogen Activator, comprising the steps of:
(a) growing, under suitable nutrient conditions, host cells transformed or transfected with an isolated DNA sequence selected from the group consisting of (i) the DNA sequences set out in SEQ ID NO:1 and SEQ ID NO:2, or (ii) DNA sequences which hybridize under stringent conditions to the DNA sequences defined in (i) their complementary strands; and
(b) isolating said recombinant Tissue Plasminogen Activator product therefrom.

2. A process for the preparation of an in vivo biologically active Tissue Plasminogen Activator product comprising the steps of transforming a host cell with a synthesized DNA sequence represented in SEQ ID NOs: 1 or 2, encoding Tissue Plasminogen Activator, and isolating said product from the host cell or the medium of its growth.

3. The process according to claim l wherein the host cells are mammalian cells.

4. The process according to claim 1 wherein the host cells are CHO K1 cells.

5. A process for the production of a soluble form of Tissue Plasminogen Activator having the in vivo biological property of treating heart attack and stroke, comprising the steps of:
a) growing, under suitable nutrient conditions, mammalian cells comprising promoter DNA, other than tissue plasminogen activator promoter DNA, operatively linked to DNA encoding the mature erythropoietin amino acid sequence of SEQ ID NO:3; and
b) isolating glycosylated erythropoietin polypeptide expressed by the cells.

6. The process of claim 5 wherein the promoter DNA is a viral promoter DNA.

7. A process for the preparation of an in vivo biologically active Tissue Plasminogen Activator product comprising the steps of transforming a host cell with a vector construct of FIG. 7 or 8 and isolating said Tissue Plasminogen Activator product from the host cell or the medium of its growth.

8. The process of claim 7, wherein the vector is a mammalian cell specific expression vector as represented in FIG. 7 or 8.

9. A pharmaceutical composition comprising a therapeutically effective amount of human Tissue Plasminogen Activator and a pharmaceutically acceptable diluent, adjuvant or carrier, wherein said Tissue Plasminogen Activator is purified from mammalian cells grown in culture.

10. The process according to claim 2 wherein the host cells are mammalian cells.

11. The process according to claim 2 wherein the host cells are CHO K1 cells.


SEQUENCE LISTING (2 sequences)

SEQ ID NO: 1 - DNA, 1687 bp, Human
  exon (1)..(1687)
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcaattggagcacagcggagagtggcgccgagtgcaccaactggcaaagcagcgcgttggcccagaagccctacagcgggcggaggccagatgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaatgtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccgcggccgcggcgtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgt

SEQ ID NO: 2 - DNA, 1689 bp, Human
  exon (1)..(1689)
atggatgccatgaagagaggtctgtgctgcgtcttgctgctgtgcggagcggtcttcgtgtccccctcccaggaaatccacgcaaggttcaggcggggcgcccggtcgtatcaggtcatctgccgtgatgagaagacccagatgatctaccagcagcaccaatcctggctgagacccgtcctgaggtccaaccgggtggagtactgttggtgtaacagtggtcgagcccaatgccattccgttcccgtgaagagctgttccgagccccgctgcttcaacggcggcacatgtcagcaggctctttacttttcagatttcgtgtgccaatgtcctgaaggcttcgccggcaagtgctgtgagatcgacacacgcgcgacatgttacgaggatcaggggatatcctaccgcggtaactggtcgacggcagagtccggagccgaatgtaccaactggcagagttccgccctggcgcagaagccatactcggggcgccggccagacgccatccgcctgggcctaggcaaccacaactactgtcgaaaccccgacagggactccaagccctggtgttacgtcttcaaggcaggtaagtactcctccgagttctgctctaccccagcctgctcggaaggtaattctgactgctattttggtaacggcagtgcctaccgcggcacgcactccctgacagagtccggagcctcatgcctgccatggaactccatgatattaatcggcaacgtctacaccgcccagaacccgagcgcgcaggccctgggcctcggcaagcacaactactgtcggaatcctgacggggacgcaaaaccatggtgccacgtcttgaagaaccgccgcctcacatgggagtactgcgacgtgccctcgtgttcgacctgcggactcagacagtactcgcagccccagttccggatcaaaggaggcttattcgccgatatcgcttcgcacccctggcaagccgccatcttcgcagccgcggccgcgtcccccggggaacgcttcctgtgcggtggcatcctgatcagtagttgctggatcctgtcagcggcccactgcttccaggagaggtttcccccacaccacctgactgtcatcctgggaagaacctaccgcgtggtgccaggggaagaggagcagaaattcgaagtggagaagtacattgtgcataaggaattcgacgacgacacgtacgacaacgacatcgccttgctgcagctgaagtcggacagctcccgctgcgcccaagaatcgtccgtggttaggacggtgtgcctcccccctgctgacctgcagctgccggactggacggagtgtgaactgtcggggtacggcaagcacgaggcgctctccccattctacagcgagcgcctcaaggaagcccacgtgcgcctgtaccccagttccaggtgcacctctcagcacttgctgaaccgcactgttaccgacaatatgctgtgtgccggtgataccaggtccgggggccctcaggccaatctgcatgacgcgtgccagggggactccggcgggcccctggtgtgtttgaacgatggaaggatgaccctggtcgggatcatctcttggggcctgggctgcggccagaaggatgtgccaggcgtctacaccaaggtgacgaactacctggactggattcgcgacaacatgaggccctga